(19) United States
(12) Patent Application Publication
(10) Pub. No.: US 2009/0004258 A1
(43) Pub. Date: Jan. 1, 2009

(54) Drug Release from Thermosensitive Liposomes by Applying an Alternative Magnetic Field

(75) Inventors: Chung-Shi Yang, Taichung City (TW); Leu-Wei Lo, Xindian City (TW); Lin-Ai Tai, Sinpu Township (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: National Health Research Institutes, Zhu Nan Chen (TW)

(21) Appl. No.: 12/129,910

(22) Filed: May 30, 2008

Related U.S. Application Data
(60) Provisional application No. 60/946,532, filed on Jun. 27, 2007.

(51) Int. Cl. A61K 9/127

(52) U.S. Cl. 424/450

(57) ABSTRACT

Thermosensitive liposomes encapsulating paramagnetic iron oxide nanoparticles are used as a drug controlled release system. Paramagnetic iron oxide nanoparticles are used to generate heat by applying alternative magnetic field to cause leakage of drugs in the liposomes.

### CROSS-REFERENCE TO RELATED APPLICATION

This application claims the priority benefit of U.S. Provisional Application Ser. No. 60/946,532, filed Jun. 27, 2007, the full disclosures of which are incorporated herein by reference.

### BACKGROUND

1. Field of Invention

The present invention relates to a drug release system. More particularly, the present invention relates to a drug controlled release system.

2. Description of Related Art

Liposome is a FDA-approved clinical-used nano-vehicle, which had been developed for over 30 years and used in clinic for over 10 years. The targeting delivery of liposome and distribution of liposomal vehicle in vivo can be controlled by size and surface modification.

Different approaches have been used to produce thermosensitive liposomes for controlled release, such as using the phase transition property of the constituent lipids [G. R. Anyarambhatla, D. Needham, Enhancement of the phase transition permeability of DPPC liposomes by incorporation of MPPC: a new temperature-sensitive liposome for use with mild hyperthermia, Journal of Liposome Research 9(4) (1999) 491-506]. For example, dipalmitoyl-phosphatidylcholine (DPPC) having a phase transition temperature of 42.5° C. is the most notable lipid. In order to reduce the drug leakage from these liposomes, cholesterol is commonly added as a lipid component. The addition of cholesterol reduces the thermal sensitivity of DPPC in cholesterol-containing liposomes. This technique has met with various degrees of success [G. R. Anyarambhatla, D. Needham, Enhancement of the phase transition permeability of DPPC liposomes by incorporation of MPPC: a new temperature-sensitive liposome for use with mild hyperthermia, Journal of Liposome Research 9(4) (1999) 491-506; M. H. Gaber, K. Hong, S. K. Huang, D. Papahadjoupoulos, Thermosensitive sterically stabilized liposomes: formulation and in vitro studies on mechanisms of doxorubicin release by bovine serum and human plasma. Pharm. Res. 12 (1995) 1407-16].

Thermosensitive liposomes have been known to have the capability of encapsulating drugs and releasing these drugs into heated tissue. Recently, successful targeted chemotherapy delivery to brain tumors in animals using thermosensitive liposomes has been demonstrated [K. Kakinuma et al, “Drug delivery to the brain using thermosensitive liposome and local hyperthermia”, International J. of Hyperthermia, Vol. 12, No. 1, pp. 157-165, 1996]. Kakinuma's study was conducted by using an invasive needle hyperthermia RF antenna placed directly within the tumor to locally heat the tumor and the liposomes. The results showed that when thermosensitive liposomes are used as the drug carrier, significant drug levels were measured within brain tumors that were heated to the range of about 41-44° C. A minimal invasive targeted treatment of large tumor is also disclosed in U.S. Pat. No. 5,810,888.

However, no noninvasive way has been developed to control the drug release from thermosensitive liposomes at a non-heated target tissue.

### BRIEF DESCRIPTION OF THE DRAWINGS

The invention can be more fully understood by reading the following detailed description of the embodiment, with reference made to the accompanying drawings as follows:

FIGS. 1A and 1B are diagrams of thermosensitive liposomes containing iron oxide nanoparticels and drugs therein according to embodiments of this invention;

FIG. 2 shows TEM images of (a) synthesized DSPE-PEG₂₀₀₀-OA-IO, (b) Resovist®, (c) DSPE-PEG2000-OA-IO (3 mg Fe/mL) nanoparticles, together with CF, encapsulated in the thermosensitive liposome, (d) Resovist® (14 mg Fe/mL) nanoparticles, together with CF, encapsulated in the thermosensitive liposome;

FIG. 3 is UV-Visible spectrum of thermal sensitive liposomes encapsulating Resovist® with CF or without CF;

FIG. 4 shows temperature-dependent variation of CF release percentage of thermosensitive liposomes encapsulating CF only;

FIGS. 5A-5D are schematic diagrams showing experimental instrumentations for measuring heating effect of paramagnetic iron oxide nanoparticles induced by alternative magnetic field according to embodiments of this invention;

FIGS. 6A-6D are diagrams showing the variation of CF release percentage of thermosensitive liposomes encapsulating various compositions with or without applying alternative magnetic field (AMF);

FIG. 6E is a diagram showing the CF release percentage of thermosensitive liposomes encapsulating 100 mM CF and 20 mg Fe/mL Resovist® with or without applying alternative magnetic field (AMF);

FIGS. 7A-7B are diagrams showing dynamic-monitoring results of leaking CF from the thermosensitive liposomes, which either encapsulated or did not encapsulate paramagnetic iron oxide nanoparticles;

FIG. 8 shows the dynamic-monitoring result of the phantom test;

FIG. 9 shows the dynamic-monitoring result of the animal test;

FIG. 10 is a diagram showing the UV-Vis spectrum of thermosensitive liposomes encapsulating Resovist®, and thermosnsitive liposomes encapsulating Resovist® and HTPS before and after heating; and

FIG. 11 is a diagram showing the dynamic monitoring results of the thermosnsitive liposomes encapsulating Resovist® and HPTS with or without applying alternative magnetic field (AMF).

### DETAILED DESCRIPTION

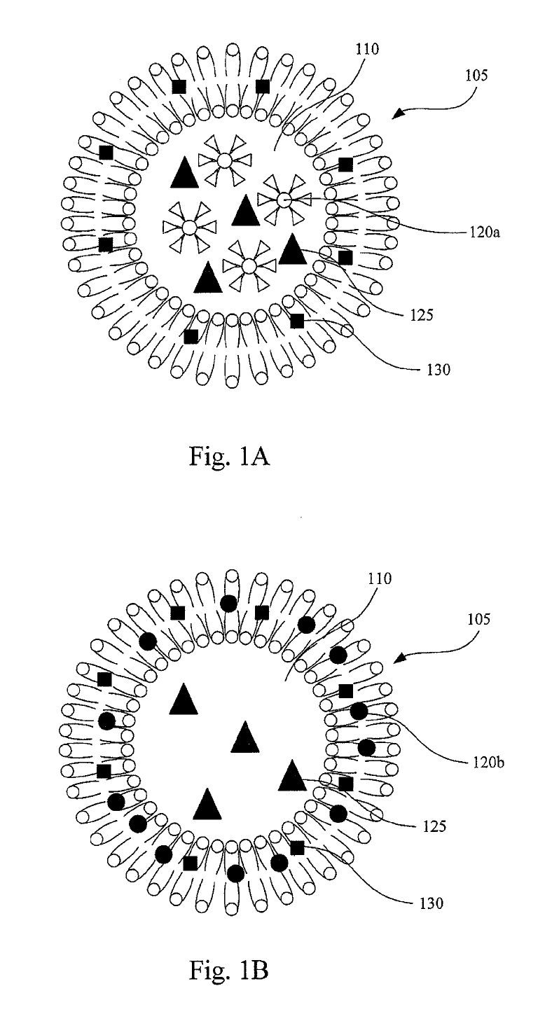

FIGS. 1A and 1B are diagrams of thermosensitive liposomes containing paramagnetic iron oxide nanoparticels and drugs therein according to embodiments of this invention. In FIGS. 1A and 1B, a thermosensitive liposome 105, composed of lipid bilayer, is used to carry hydrophilic drugs 125 in the aqueous core 110 and/or hydrophobic drugs 130 in the lipid bilayer.

In FIG. 1A, surfaces of paramagnetic iron oxide nanoparticles 120a are modified by at least a hydrophilic functional group, such as —OH, —COOH, or other suitable hydrophilic functional groups, so that the paramagnetic iron oxide nanoparticles 120a can be encapsulated in the aqueous core 110 of the thermosensitive liposomes 105. For example, the surface of the paramagnetic iron oxide nanoparticles 120a can be modified by polyethylene glycol and/or dextran. In FIG. 1B, surfaces of paramagnetic iron oxide nanoparticels 120b are not modified by any hydrophilic functional groups or modified by at least a hydrophobic functional group. Hence, the paramagnetic iron oxide nanoparticels 120b are located in the lipid bilayer of the thermosensitive liposome 105.

The thermosensitive temperature of the thermosensitive liposomes described above can be adjusted by the lipid composition. For example, the thermosensitive temperature of a thermosensitive liposome composed of DPPC (16:0 PC, Tm=41° C.), DSPC (18:0 PC, Tm=55° C.), and cholesterol can be fine tuned in a range of about 36° C. to about 53° C. (Table 1). In one embodiment of this invention, the thermosensitive temperature of the liposome is tuned to a temperature of about 2° C. to about 3° C. higher than a temperature of a target environment.

TABLE 1

| Weight ratio of lipid composition | Thermosensitive temperature (° C.) |
|---|---|
| DPPC¹: Cholesterol = 15:3 | 36-38 |
| DPPC: DSPC²: Cholesterol = 10:5:3 | 40-42 |
| DPPC: DSPC: Cholesterol = 5:10:3 | 46-48 |
| DSPC: Cholesterol = 15:3 | 51-53 |

¹DPPC: 1,2-dipalmitoyl-sn-glycero-3-phosphochloine
²DSPC: 1,2-distearoyl-sn-glycero-3-phosphocholine

The surface of the thermosensitive liposomes can be modified by some polymer to further tune the thermosensitive temperature of the thermosensitive liposomes. The examples of the polymer were disclosed in “Thermosensitive polymer-modified liposome” Kono, K., Adv. Drug. Deliv. Rev. 2001, 53, 307-319, which is incorporated here entirely by reference.

The use of paramagnetic magnetite nanoparticles (Fe₃O₄) in clinical medicine is an important field in diagnostic medicine and drug delivery. Magnetite nanoparticles, with size of 10-20 nm, are superparamagnetic. These magnetite nanoparticles can interfere with an external homogeneous magnetic field and can be positioned magnetically in a living body to facilitate magnetic resonance imaging (MRI) for medical diagnosis [U.S. Pat. No. 6,123,920, U.S. Pat. No. 6,048,515, U.S. Pat. No. 6,203,777, U.S. Pat. No. 6,207,134, which are incorporated herein by reference, D. K. Kim, et al, J. Magn. Mag. Mater., 225, 256 (2001)]. The magnetite nanoparticles can also generate heat under an alternative magnetic field (AMF) due to magnetic hysteresis loss; this phenomena is called magnetic fluid hyperthermia (MFH), which can be used for cancer therapy [U.S. Pat. No. 6,165,440, U.S. Pat. No. 6,167,313, which are incorporated herein by reference, A. Jordan, et al, J. Magn. Mag. Mater., 201, 413 (1999)].

Accordingly, the thermosensitive liposome 105, containing paramagnetic iron oxide nanoparticels 120a or 120b therein, can be heated by an AMF, a noninvasive force, to release drugs 125 and/or 130 therefrom. That is, drugs 125 and/or 130 can be released from the thermosensitive liposome 105 via a noninvasive way.

### Materials

Resovist® (Ferucarbotan, 0.5 mmol Fe/mL, MRI liver contrast agent, for injection, 1.4 mL/syringe) was purchased from Schering Diagnostics (Schering AG, Germany).

Cloroform solution of 1,2-dipalmitoyl-sn-glycero-3-phosphochloine (DPPC, M=734.05), 1,2-distearoyl-sn-glycero-3-phosphocholin (DSPC, M=790.16) and 1,2-diacyl-sn-glycero-3-phoshoethanolamine-N-[methoxy(poly (ethylene glycol))-2000] (DSPE-PEG₂₀₀₀, M=2805.54) were purchased from Avanti Polar Lipids (Alabaster, Ala.).

Cholesterol (Chol), Triton X100, ferric chloride, sodium oleate, oleic acid, 1-octadecene, hexane and ethanol were purchased from Sigma (St Louis, Mo.).

5-(and-6)-carboxylfluorescein was purchased from Invitrogen (Eugene, Oreg., USA).

Unless otherwise stated, the buffer used was 100 mM phosphate buffer solution, pH 7.0.

### Synthesis of Paramagnetic Iron Oxide Nanoparticles

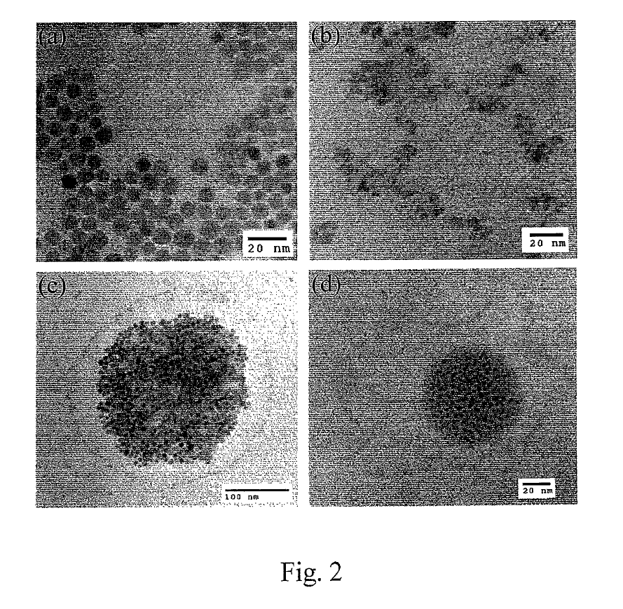

According to an embodiment of this invention, two types of paramagnetic iron oxide nanoparticles were employed. One is paramagnetic iron oxide nanoparticles coated with DSPE-PEG₂₀₀₀ (denoted as DSPE-PEG₂₀₀₀-OA-IO), which has an iron oxide core of about 8 nm and a hydrodynamic diameter of 16.2±1.7 nm. The TEM image of the synthesized DSPE-PEG₂₀₀₀-OA-IO is shown in FIG. 2(a).

The other is a commercially available dextrane-coated iron oxide nanoparticle, called Resovist®. Resovist® is a clinically used liver MRI contrast agents, which has a polycrystalline iron oxide core (3-5 nm) coated with dextrane with a hydrodynamic diameter of 43.0±7.2 nm. The TEM image of Resovist® is shown in FIG. 2(b).

The DSPE-PEG₂₀₀₀-coated paramagnetic iron oxide nanoparticles described above was synthesized by the following method. An oleic acid coated, highly hydrophobic, monodisperse paramagnetic iron oxide nanoparticles was synthesized according to a procedure already described [Park, J., An, K., Hwang, Y., Park, J. G., Noh, H. J., Kim, J. Y., Park, J. H., Hwang, N. M. & Hyeon, T. Ultra-large-scale synthesis of monodisperse nanocrystals. Nature Materials 3, 891-895 (2004). Sun, S., Zeng, H., Robinson, D. B., Raoux, S., Rice, P. M., Wang, S. X. & Li, G. Monodisperse MFe₂O₄ (M=Fe, Co, Mn) nanoparticles. J. Am. Chem. Soc. 126, 273-279 (2004)]. First, iron-oleate complex (Fe(Oleate)₃) was prepared by reacting ferric chloride (10.8 g, FeCl₃.6H₂O, 40 mmol) and sodium oleate (36.5 g, 120 mmol) in a mixture solvent composed of 80 mL ethanol, 60 mL distilled water and 140 mL hexane. The resulting solution was heated to 70° C. and kept at that temperature for four hours. When the reaction was completed, the upper organic layer containing the iron-oleate complex was wash three times with 30 mL distilled water in a separatory funnel. After wash, hexane was evaporated to obtain iron-oleate complex in waxy solid form.

Next, monodispersed iron oxide nanocrystals was prepared. 36 g (40 mmol) of the iron-oleate complex and 5.7 g of oleic acid (20 mmol) were dissolved in 200 g of 1-octadecene at room temperature. The reaction mixture was heated to 320° C. and then kept at that temperature for 30 min. The resulting solution containing the nanocrystals was then cooled to room temperature, and 500 mL of ethanol was added to the solution to precipitate the nanocrystals and were separated by centrifugation. The synthesized oleic acid coated iron oxide (OA-IO) nanocrystals was highly dissolved in organic solvent (e.g. hexane, or chloroform) without any aggregation.

The DSPE-PEG₂₀₀₀ coated OA-IO nanocrystals were synthesized by mixing 100 mg DSPE-PEG₂₀₀₀ and 200 mg OA-IO crystals in chloroform using a micelle formation protocol [Dubertret, B., Skourides, P., Norris, D. J., Noireaux, V., Brivanlou, A. H. & Libchaber, A. In vivo imaging of quantum dots encapsulated in phospholipid micelles. Science 298, 1759-1762 (2002), which is incorporated by reference]. Following evaporating the chloroform in a 60° C. water bath, a thin film was formed and then dried overnight under vacuum. The film was hydrated in 100 mL 60° C. distilled water to form the DSPE-PEG₂₀₀₀ coated OA-IO (DSPE-PEG₂₀₀₀-OA-IO) nanocrystals. The DSPE-PEG₂₀₀₀-OA-IO nanocrystals, which was highly dissolved in water, was further purified by 100 nm filter and centrifugation.

### Preparation of Thermosensitive Liposome Encapsulating Iron Oxide Nanoparticles and CF

### Method 1

The thermosensitive temperature of thermosensitive liposome is tunable by changing the lipid composition. In this embodiment, DPPC, DSPC and Chol were used to compose the thermosensitive liposomes. Two compositions were synthesized here, DPPC: Chol=5:1 by weight and DPPC:DSPC:Chol=10:5:3 by weight. The preparation method of thermosensitive liposomes, composing of DPPC: Chol=5:1 by weight, encapsulating various paramagnetic iron oxide nanoparticles are described by examples as follow.

Thermosensitive liposomes containing paramagnetic iron oxide nanoparticles therein were prepared by a thin film hydration method coupled s with sequential extrusion. Aliquot of DPPC: Chol=5:1 by weight (total lipid 10 mg) in chloroform was placed into a round bottom flask and heated at a temperature higher then the highest melting temperature of the composed lipids (here is 50° C.) in water bath. At the same time, the chloroform was removed to form a dry film of lipids in the flask by rotary evaporator under vacuum for 12 h.

Dry film of lipids was hydrated by adding 1 mL aqueous suspension of paramagnetic iron oxide nanoparticles with various surface modifications (3 mg Fe /mL DSPE-PEG₂₀₀₀-OA-IO, 7 mg Fe/mL Resovist®, or 14 mg Fe/mL Resovist®) and 100 mM aqueous solution of carboxylfluorescein (CF), and the mixture was then incubated in 50° C. water bath for 30 min. Dispersions were homogenized with mini-extruder at 50° C. through 400 nm polycarbonate filters (Avanti Polar Lipids, Alabaster, Ala.). Non-encapsulated CF was removed by sephadex G-25 size exclusion column first, and the non-encapsulated paramagnetic iron oxide nanoparticles was removed by filtration through 0.1 μm Amicon low-binding Durapore PVDF membrane (Millipore Corporation, Bedford, Mass.) using centrifugation at 2000 rpm.

For example, TEM images of DSPE-PEG2000-OA-IO nanoparticles (3 mg Fe/mL), together with CF, encapsulated in the thermosensitive liposome (DPPC: Chol=5:1 by weight) and Resovist® nanoparticles (14 mg Fe/mL), together with CF, encapsulated in the thermosensitive liposome (DPPC: Chol=5:1 by weight) are shown in FIGS. 2(c) and 2(d), respectively. The hydrodynamic diameter of the liposomes in FIGS. 2(c) and 2(d) was about 300-450 nm.

Hydrodynamic diameters of paramagnetic iron oxide nanoparticles and liposomes described above were determined by a particle size analyzer (90 plus particle size analyzer, Brookhaven Instruments Corporation, Long Island, USA).

### Method 2

In this embodiment, thermal sensitive liposome (DPPC: DSPC: Chol=14:1:3 by weight) encapsulating Resoviste (20 mg Fe/mL) only was synthesized according to method 1 above but without adding CF at all. Then, tangential flow filtration system (TFF system) was used to remove the un-trapped iron oxide nanoparticles by using 0.1 μm Durapore filter cassette.

Next, the purified thermal sensitive liposome encapsulating Resovist® was mixed with 100 mM of CF in PBS buffer system (pH 7.4). Then, the mixture was extruded by a filter at a temperature higher than the thermosensitive temperature of the thermal sensitive liposomes to load CF into the thermal sensitive liposome. When the temperature is higher than the thermosensitive temperature of this thermal sensitive liposome in this embodiment, the membrane of liposome becomes permeable for CF but not permeable for Resovist®. The size of the thermal sensitive liposome was decided by the pore size of the extrusion polycarbonate filter used.

Finally, the CF outside the thermal sensitive liposomes was removed by Sephadex G-25 size exclusion column.

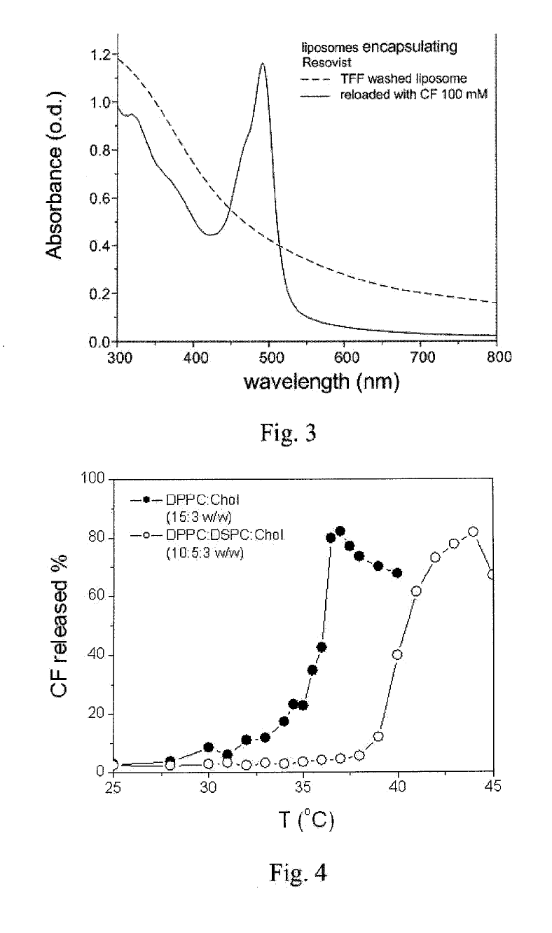

FIG. 3 is UV-Visible spectrum of thermal sensitive liposomes encapsulating Resovist® with CF or without CF. In FIG. 3, the thermal sensitive liposomes encapsulating Resovist® and CF showed an obvious absorption peak at 420-520 nm, which resulted from CF. This absorption peak demonstrated the efficient loading of CF into the thermal sensitive liposomes.

Using method 2 to prepare thermosensitive liposomes encapsulating iron s oxide nanoparticles and drugs can avoid drugs lost during the purification processes. In addition, the variables of loading drugs into the thermosensitive liposomes can be controlled more easily. Moreover, the size of the purified thermosensitive liposomes encapsulating iron oxide nanoparticles and drugs can be further confined after the extrusion step. For example, the size of purified thermal sensitive liposomes encapsulating Resoviste was about 600-1000 nm. After encapsulating the CF and extruded by a 200 nm PC membrane, the size was reduced to 230-320 nm.

### Monitoring the Thermosensitivity of the Prepared Liposome

The leakage of thermosensitive liposome was monitored by the encapsulated fluorescence dye, CF. The fluorescence of CF is self-quenched at a high concentration, such as at a concentration of about 100 mM. Therefore, no fluorescence was generated when CF was encapsulated in liposome. Since the prepared liposomes were theromsensitive liposomes, CF can be released from the liposomes when the environment temperature was over the thermosensitive temperature of liposome to increase the fluorescence intensity.

Water bath or water circulator was used to control the environment temperature. The thermosensitive temperatures of the thermosensitive liposomes were measured by incubating in water bath for 30 min at various temperatures. The fluorescence intensity of CF (excitation at 480 nm, emission at 516 nm) was monitored as an indicator of liposomal leakage. Standard sample was prepared by using Triton X100 to cause liposome lysis to release all of the encapsulated CF. The release percentage of CF can be calculated by the fluorescence intensity of each sample.

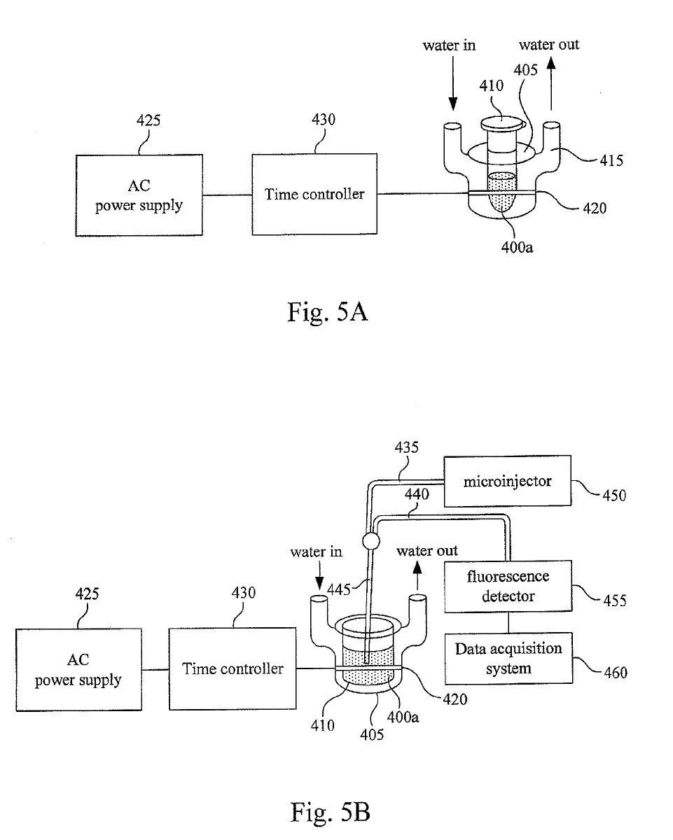

FIG. 4 shows temperature-dependent variation of CF release percentage of thermosensitive liposomes encapsulating CF only. In FIG. 4, Solid circle represents the data obtained from the thermosensitive liposomes having the first composition of DPPC: Chol=5:1 by weight, and the open circle represents the data obtained from the thermosensitive liposomes having the second composition of DPPC:DSPC:Chol=10:5:3 by weight. It can be seen from FIG. 3 that when the temperature reach the thermosensitive temperature of the liposomes, the fluorescence intensity of CF was greatly increased.

Since the melting temperature, 55° C., of DSPC is higher than the melting point, 42° C., of DPPC, the thermosensitive temperature, 40-42° C., of the thermosensitive liposomes having the second composition is higher than the thermosensitive temperature, 35-37° C., of the thermosensitive liposomes having the first composition.

### Heating Effect of Paramagnetic Iron Oxide Nanoparticles Induced By Alternative Magnetic Field

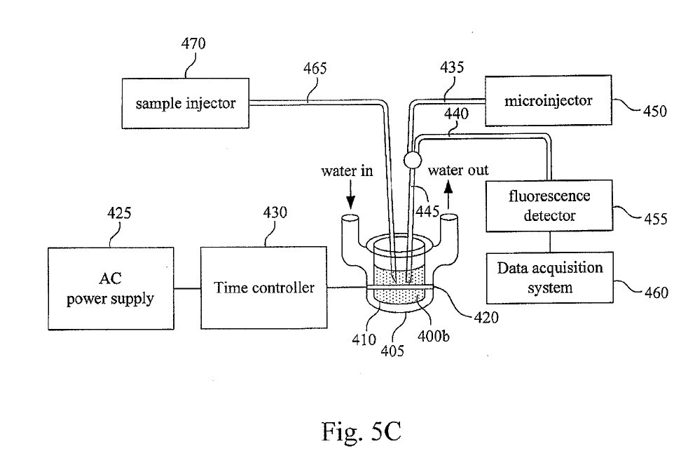

FIGS. 5A-5D are schematic diagrams showing experimental instrumentations for measuring heating effect of paramagnetic iron oxide nanoparticles induced by alternative magnetic field according to embodiments of this invention.

In FIG. 5A, a water jacket 405 was used to hold a sample holder 410, such as a 200 μL Appendove tube, therein, and a water circulator 415 was used to isolate the heat generated from an induction coil 420 (2 cycles for small coil having a diameter of 1.5 cm, and one cycle for big coil having a diameter of 3.0 cm) surrounding the water jacket 405 and control the environment temperature of samples. The induction coil 420 was used to generate an alternative magnetic field surrounding the water jacket 405. An alternative current (AC) power supply (Power cube 64/900, 750-1150 KHz, 6.4 kW, Ceia Company, Arezzo, Italy) 425 of the solid-state high frequency generator type was used to supply electric power to the induction coil 420. A time controller 430 was optionally connected between the induction coil 420 and the AC power supply 425.

The sample bottles 410 were used to load thermosensitive liposomal solutions (40 μL) 400a, either encapsulating or not encapsulating paramagnetic iron oxide nanoparticles. The leakage of the encapsulated CF in the thermosensitive liposomes, with or without applying the AMF, were monitored by the fluorescence intensity of CF. All samples were incubated in the water jacket 405 at a temperature of about 28-38° C. for 30 min with applying AMF at 5-25 minutes or without applying AMF.

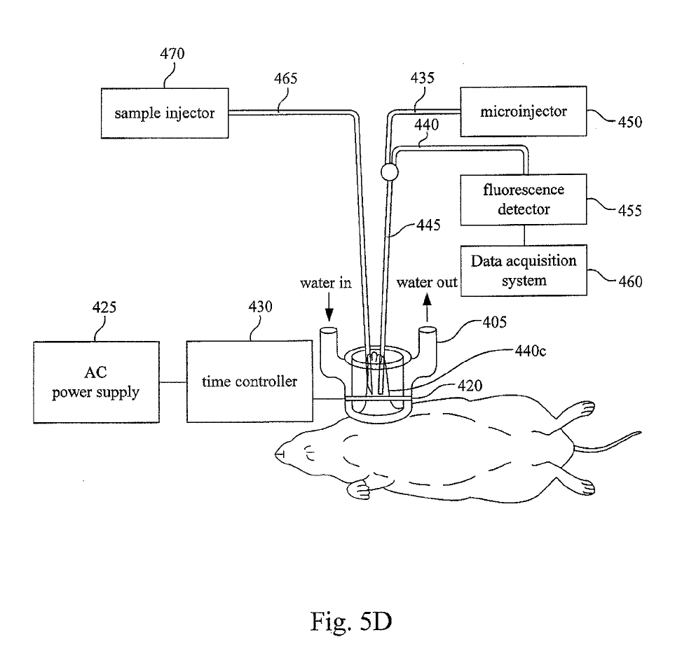

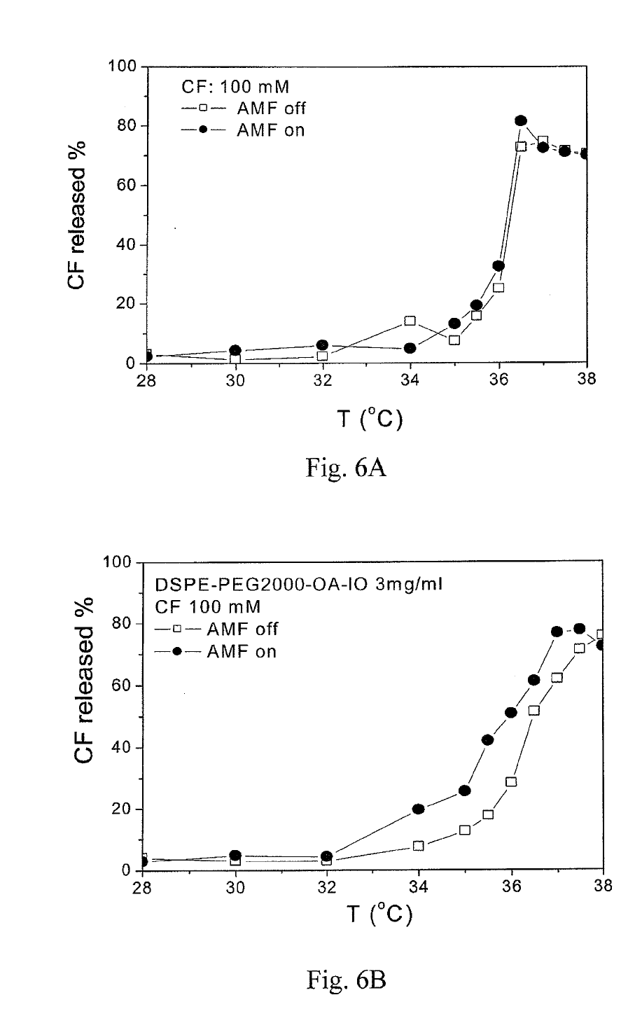

FIGS. 6A-6D are diagrams showing the variation of CF release percentage of thermosensitive liposomes encapsulating various compositions with or without applying alternative magnetic field (AMF). In FIGS. 6A-6D, the thermosensitive liposomes had the composition of DPPC:Chol=5:1 by weight. In FIG. 6A, the thermosensitive liposomes encapsulated 100 mM carboxylfluorecein (CF) only. The thermosensitive temperatures of thermosensitive liposomes (about 35-37° C.) were almost the same with or without applying AMF.

In FIGS. 6B-6D, the thermosensitive liposomes encapsulating 100 mM CF and 3 mg Fe/mL DSPE-PEG₂₀₀₀-OA-IO, 100 mM CF and 7 mg Fe/mL Resovist®, and 100 mM CF and 14 mg Fe/mL Resovist®, respectively, were prepared by the method 1 described above. The thermosensitive temperature differences between with and without applying AMF were about 0.5° C., 1.5° C., and 4.0° C., respectively.

FIG. 6E is a diagram showing the CF release percentage of thermosensitive liposomes encapsulating 100 mM CF and 20 mg Fe/mL Resovist® with or without applying alternative magnetic field (AMF). In FIG. 6E, the thermosensitive liposomes encapsulating 100 mM CF and 20 mg Fe/mL Resovist® was prepared by the method 2 described above. The samples were incubated at a temperature of about 32-39° C. for 30 min with applying AMF at 5-30 minutes or without applying AMF. The thermosensitive temperature difference between with and without applying AMF was about 2-3° C.

It demonstrated that the internal thermal sources (i.e. paramagnetic iron oxide nanoparticles here) can response to the external AMF to heat the liposome inside, and the encapsulated CF was hence leak from the thermosensitive liposomes to increase the fluorescence intensity. The amount of heat generated was related to the concentration of the paramagnetic iron oxide nanoparticles encapsulated in the liposomes.

On-Line and Dynamic Monitoring of CF Release from Thermosensitive Liposomes

Dynamic monitoring the leakage of carboxylfluorescein from liposomes was performed by microdialysis. In FIG. 5B, a microdialysis probe 445, connect with a microinjector 450 via a buffering tube 435 and a fluorescence detector 445 via a sampling tube 440 was placed in the liposomal solution 400a in the sample holder 410 held by the water jacket 405. The fluorescence detector 455 was used to monitor the microdialsylate, the leaked carboxylfluorescein, at a frequency of 1 Hz. The obtained fluorescence data was collected by a data acquisition system 460 connected to the fluorescence detector 455. The microinjector 450 was used to deliver 100 mM phosphate buffer solution (pH 7.0) via the buffering tube 435 to the liposomal solution 400a at a flow rate of 1 μL/min.

The light source of the fluorescence detector 455 is a 488-nm argon laser. The fluorescence detector 455 is a photon multiplier tube (PMT). A lag time from the microdialysis probe 445 to a fluorescence detector 455 is about 11 min due to the connection loop between the microdialysis probe 445 and the fluorescence detector 455.

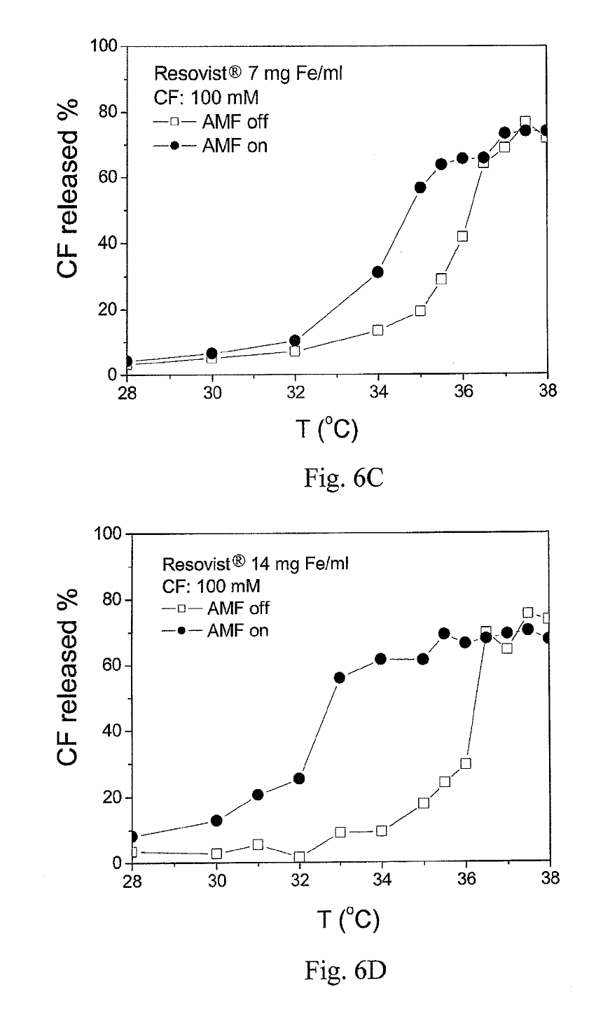

FIGS. 7A-7B are diagrams showing dynamic-monitoring results of leaking CF from the thermosensitive liposomes, which either encapsulated or did not encapsulate iron oxide nanoparticles. The thermosensitive liposomes, having a thermoensitive temperature of 35-37° C., either encapsulating or without encapsulating Resovist (14 mg Fe/mL) were incubated in the water jacket at 33.5° C.

In FIGS. 7A and 7B, a significant increase of PMT voltage was observed after applying AMF with a lag time of about 14 min for the sample of Resovist® encapsulated by thermosensitive liposome. The lag time, about 14 min, was a sum of the system delay time, about 11 min, and the time needed to generate heat to reach the thermosensitive temperature of the liposomes. Hence, the time needed to generate heat to reach the thermosensitive temperature of the liposomes was about 3 min. Contrarily, the blank sample without encapsulating paramagnetic iron oxide nanoparticles did not response to the applied AMF at all.

In FIG. 7A, the water jacket was surrounded by 2 cycles of 1.5-cm induction coil. In FIG. 7B, the water jacket was surrounded by 1 cycle of 3.0-cm induction coil. It shows that the induction coil having higher cycle number can induce more fluorescence intensity.

### Thermosensitive Release of Liposomes in Phantom

In vitro gel phantom (1% agarose solution) was used to mimic the non-homogeneous environment in vivo. In FIG. 4C, a sample injector 460 of a needle type was used to inject liposomal solution prepared above via a delivering tube 465 into the gel phantom 400b in the sample holder 410 held by the water jacket 405 at a flow rate of 5 μL/min. The other equipment shown in FIG. 4C was basically the same as those shown in FIG. 5B.

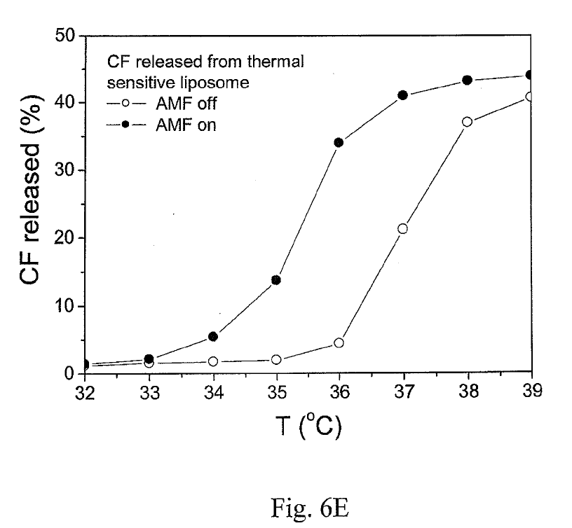

FIG. 8 shows the dynamic-monitoring result of the phantom test. Similar results were observed in the phantom system, the fluorescence dye was significantly released in the sample of Resovist® encapsulated by thermosensitive liposome and no response in blank samples without encapsulating paramagnetic iron oxide nanoparticles. Repeatedly applying AMF showed controllable release of drugs.

### Thermosensitive Release of CF in Animal

Skeletal muscle of a rat forearm was used as an in vivo model to monitor the release of fluorescence dye from thermosensitive liposome encapsulating paramagnetic iron oxide nanoparticles by applying AMF.

In FIG. 5D, the sample holder 410 in FIG. 5B were omitted, and a rat forearm 440c was placed in a hollow water jacket 405 at 33.5° C. The liposomal solutions were also introduced to the rat forearm 440c by the sample injector 470 via the delivering tube 465 at a flow rate of 5 μL/min. The delivering tube 465 was co-implanted with the microdialysis probe 445 into the skeletal muscle of the rat forearm 400c. The water jacket 405 was surrounded by 1 cycle of 3.0-cm induction coil 420.

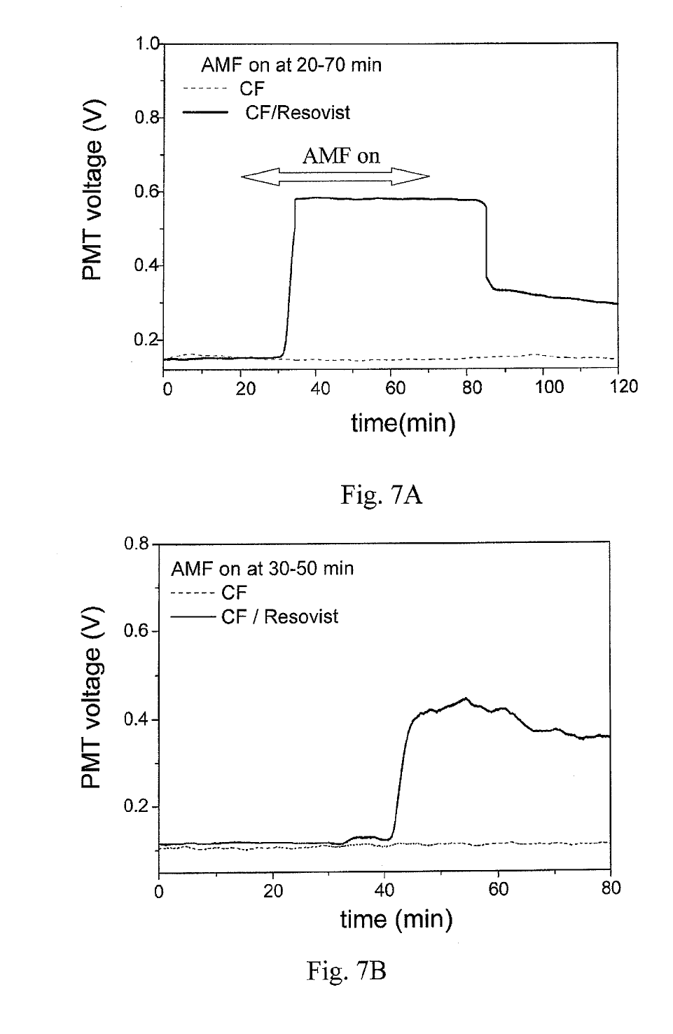

In this in vivo system, Resovist® (14 mg Fe/mL) was encapsulated by the thermosensitive liposomes. FIG. 9 shows the result of the animal test. The result was similar to the results stated above (FIGS. 7A, 7B and 8).

### Preparation of Thermosensitive Liposome Encapsulating Iron Oxide Nanopartcicles and HPTS

In this embodiment, HPTS (8-hydroxy-1,3,6-pyrenetrisulfonic acid) was used to replace the CF above. HPTS is a pH sensitive dye. The UV-Vis absorption spectrum is varied when the pH of the environment is changed. Hence, the UV-Vis spectrum can be used to monitor the environmental pH change of the HPTS.

For example, HPTS was first loaded into the thermosensitive liposomes encapsulating Resovist® at a basic condition (pH 10), and free HPTS was then removed. Next, the thermosensitive liposomes encapsulating Resovist® and HPTS were transferred to a neutral environment (pH 7) and then heated at a temperature higher then the thermosensitive temperature of the thermosensitive liposomes.

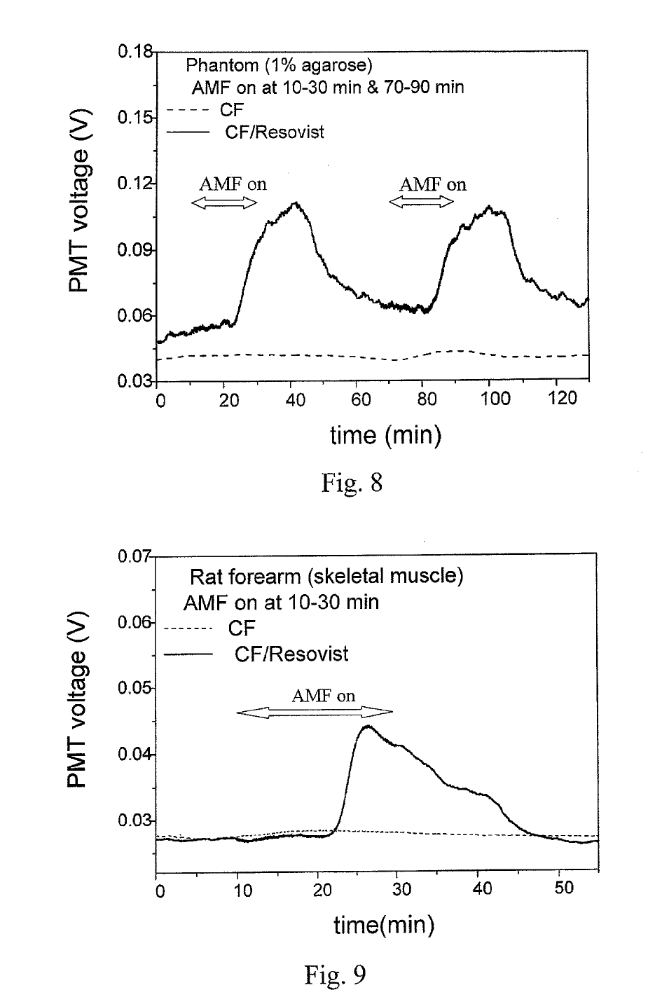

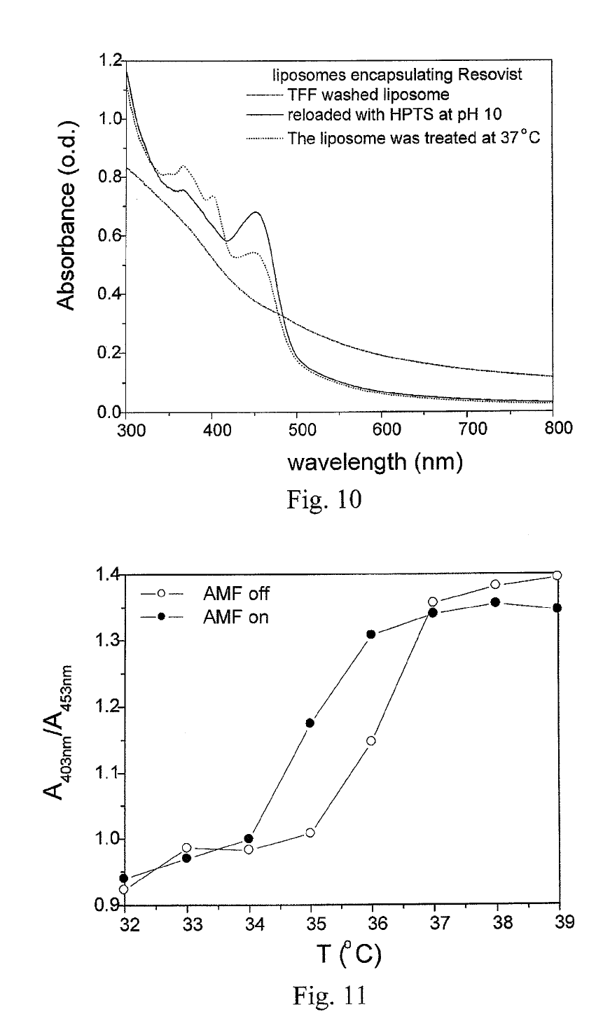

Measuring the UV-Vis spectrum before and after heating, an absorption peak at 453 nm was decreased while an absorption peak at 403 nm was increased, as shown in FIG. 10. The A₄₀₃ₙₘ/A₄₅₃ₙₘ is further used to monitor the thermosensitive temperature of the thermosensitive liposomes with or without applying AMF, as shown in FIG. 11. All samples were incubated at a temperature of 32-39° C. for 30 minutes with applying AMF at 5-30 minutes or without applying AMF. In FIG. 11, the thermosensitive temperature difference of the thermosensitive liposomes with or without applying AMF was about 2° C.

It showed that a pH gradient can be established across the lipid bilayer of the thermosensitive liposomes. Therefore, the thermosensitive liposomes can also be used to create a micro-environment for some drugs that can only stable at a certain condition, and the drugs are then controlled release by applying AMF after delivering to a target site.

Accordingly, a drug controlled release system by a noninvasive force is disclosed. Paramagnetic iron oxide nanoparticles are encapsulated in thermosensitive liposomes and used to generate heat by applying alternative magnetic field. Since the paramagnetic iron oxide nanoparticles are encapsulated in thermosensitive liposomes, the concentration of the paramagnetic iron oxide nanoparticles can easily reach the minimum required concentration (about 10 mg Fe/mL) to effectively generate heat without using large amount of paramagnetic iron oxide nanoparticles to avoid possible toxicity when used in vivo. Moreover, the thermosensitive temperature of liposomes is variable by adjusting the composition of lipids, and the thermosensitive temperature is hence preferably adjusted to a temperature higher than the environmental temperature for at least about 2-3° C. For example, if the environment is in a human body having a temperature of about 37° C., the thermosensitive temperature of the liposomes is preferably adjusted to about 39-40° C.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A composition for thermally-controlled drug release by an alternative magnetic field (AMF), comprising:
thermosensitive liposomes for carrying a drug; and
paramagnetic iron oxide nanoparticels in the thermosensitive liposomes, so that the paramagnetic iron oxide nanoparticels can be heated by the AMF to cause leakage of the thermosensitive liposome in an target environment.

2. The composition of claim 1, wherein the thermosensitive temperature of the thermosensitive liposomes is about 2° C. to about 3° C. higher than the temperature of the target environment.

3. The composition of claim 1, wherein the composition of the thermosensitive liposomes is Cholesterol and a lipid selected from a group consisting of DPPC, DSPC, and a combination thereof.

4. The composition of claim 1, wherein the surface of the paramagnetic paramagnetic iron oxide nanoparticles are chemically modified by hydrophilic moiety.

5. The composition of claim 4, wherein the hydrophilic moiety is PEG or dextran.

6. The composition of claim 1, wherein the surface of the paramagnetic paramagnetic iron oxide nanoparticles are chemically modified by hydrophilic functional group.

7. The composition of claim 6, wherein the hydrophilic functional group is —OH, —COOH, or a combination thereof.

8. A method of delivering a drug to a target site in a subject, comprising:
providing thermosensitive liposomes, which contains paramagnetic iron oxide nanoparticles and a drug, in the target site; and
applying an alternative magnetic field to the target site, so that the paramagnetic iron oxide nanoparticels can be heated by the AMF to cause the drug to be released by the thermosensitive liposomes in the target site.

9. The method of claim 8, wherein the thermosensitive temperature of the thermosensitive liposomes is about 2° C. to about 3° C. higher than the temperature of the target site.

10. The method of claim 8, wherein the composition of the thermosensitive liposomes is Cholesterol and a lipid selected from a group consisting of DPPC, DSPC, and a combination thereof.

11. The method of claim 8, wherein the surface of the paramagnetic iron oxide nanoparticles are chemically modified by hydrophilic moiety.

12. The method of claim 8, wherein the hydrophilic moiety is PEG or dextran.

13. The method of claim 8, wherein the surface of the paramagnetic iron oxide nanoparticles are chemically modified by hydrophilic functional group.

14. The method of claim 13, wherein the hydrophilic functional group is —OH, —COOH, or a combination thereof.
